(19) United States
(12) Patent Application Publication
(10) Pub. No.: US 2013/0191075 A1
(43) Pub. Date: Jul. 25, 2013

(54) Status Tracking Method and Apparatus for Diagnostic Testing

(71) Applicant: Service Solutions U.S. LLC, Warren, MI (US)

(72) Inventors: Olav M. Underdal, San Diego, CA (US); Harry M. Gilbert, Portage, MI (US); Alex Portyanko, Kalamazoo, MI (US); Randy L. Mayes, Otsego, MI (US); Gregory J. Fountain, Kalamazoo, MI (US); William W. Wittliff, III, Gobles, MI (US)

(73) Assignee: Service Solutions U.S. LLC, Warren, MI (US)

(21) Appl. No.: 13/794,039

(22) Filed: Mar. 11, 2013

Related U.S. Application Data
(63) Continuation-in-part of application No. 13/084,253, filed on Apr. 11, 2011, now Pat. No. 8,412,402.
Application No. 13/084,253 is a continuation of application No. 12/651,745, filed on Jan. 4, 2010, now Pat. No. 7,925,397.
Application No. 12/651,745 is a continuation of application No. 11/452,243, filed on Jun. 14, 2006, now Pat. No. 7,643,916.

(51) Int. Cl. G06F 11/32; A61B 5/00

(52) U.S. Cl. CPC G06F 11/321; A61B 5/00; A61B 5/742; A61B 5/486
USPC 702/183; 600/300

(57) ABSTRACT

A status tracker tracks the state of a patient during a diagnostic test sequence to eliminate redundant steps between diagnostic procedures. The status tracker maintains a list of preconditions required for each diagnostic procedure, reads a current patient status from a memory register and verifies the current setting of the patient status corresponding to a specific precondition. If the precondition is required and the current setting is not valid, the state tracker further formats a test preparation step corresponding to the precondition for display to instruct a patient technician to satisfy the precondition. Otherwise, if the precondition is not required and the corresponding setting is valid, the status tracker formats an instruction to reverse the corresponding patient status or condition. The status tracker additionally receives feedback indicating when a precondition has been satisfied or the corresponding patient status has been reversed, and updates the patient status in memory.

### CROSS REFERENCE TO RELATED APPLICATIONS

This application claims the benefit of and is a continuation-in-part of U.S. patent application Ser. No. 13/084,253, filed Apr. 11, 2011, which is a continuation of U.S. patent application Ser. No. 12/651,745, filed Jan. 4, 2010, now U.S. Pat. No. 7,925,397, which is a continuation of U.S. patent application Ser. No. 11/452,243, filed Jun. 14, 2006, now U.S. Pat. No. 7,643,916, entitled “Operational state Tracking Method and Apparatus for Diagnostic Testing,” all of which are hereby incorporated by reference in their entireties.

### FIELD OF THE INVENTION

The present invention relates generally to diagnostic equipment. More particularly, the present invention relates to tracking a status of a patient, a vehicle, an appliance, and/or a power tool during a diagnostic test sequence.

### BACKGROUND OF THE INVENTION

Diagnostic systems are used by technicians and professionals in virtually all industries to perform basic and advanced system testing functions. For example, in the medical, automotive, appliance, trucking, heavy equipment and aircraft industries, diagnostic test systems provide for onboard computer fault or trouble code display, interactive diagnostics, multiscope and multimeter functions, and electronic service manuals. In the medical industry, diagnostic systems provide for monitoring body functions and diagnosis of medical conditions, as well as system diagnostics to detect anomalies in the medical equipment.

In many industries, diagnostic systems play an increasingly important role in manufacturing processes, as well as in maintenance and repair throughout the lifetime of the patient, equipment or product. Some diagnostic systems are based on personal computer technology and feature user-friendly, menu-driven diagnostic applications. These systems assist technicians and professionals at all levels in performing system diagnostics on a real-time basis.

A typical diagnostic system includes a display on which instructions for diagnostic procedures are displayed. The system also includes a system interface that allows the operator to view real-time operational feedback and diagnostic information. Thus, the operator may view, for example, vehicle or machine engine speed in revolutions per minute, or battery voltage during start cranking; or a patient's heartbeat rate or blood pressure. With such a system, a relatively inexperienced operator may perform advanced diagnostic procedures and diagnose complex operational or medical problems.

The diagnostic procedures for diagnostic systems of this sort are typically developed by experienced technical experts or professionals. The technical expert or professional provides the technical experience and knowledge required to develop complex diagnostic procedures. Thus, the efficacy of the diagnostic procedures, in particular the sequence in which the diagnostic procedures are performed, is highly dependent on the expertise of the technical expert or professional authoring the procedures.

Some existing diagnostic systems have a disadvantage in that each diagnostic procedure in a sequence of diagnostic procedures includes test preparation steps without regard to the preceding diagnostic test procedures. As a result, when performing the diagnostic procedures, the vehicle or machine technician may return the vehicle or machine to a default starting configuration at the end of an individual diagnostic procedure, only to realize that the following test procedure requires one or more of the same test preparation steps. This process can result in the expenditure of unnecessary time, cost and duplication of effort. Accordingly, it is desirable to provide a method and apparatus for tracking the state of a vehicle or machine during a sequence of diagnostic test procedures in a format that can be executed on a PC-based diagnostic system.

### SUMMARY OF THE INVENTION

The foregoing needs are met, to a great extent, by the present invention, wherein in one aspect an apparatus and method are provided that in some embodiments can track the status of a patient, appliance, or power tool during a sequence of diagnostic test procedures in a format that can be executed on a PC-based diagnostic system.

An embodiment of the present invention pertains to a medical diagnostic tool for use with a medical diagnostic test. The medical diagnostic tool includes a precondition determiner, a setting verifier, and a preparatory formatter. The precondition determiner is configured to determine if a precondition is required for a subsequent medical diagnostic test. The setting verifier is configured to verify a current state of the precondition for the subsequent medical diagnostic test. The preparatory formatter is configured to provide instructions to make the precondition ready if required for the subsequent medical diagnostic test if the precondition is not ready for the subsequent medical diagnostic test.

Another embodiment of the present invention relates to a computer implemented method of tracking a state of a precondition. In this method, the precondition required for a subsequent medical diagnostic test is determined via a processor of the computer. In addition, it is determined if the required precondition is ready for the subsequent medical diagnostic test, via the processor and instructions are provided to a user to make the required precondition ready if the precondition is not ready, via the processor.

Yet another embodiment of the present invention pertains to a machine diagnostic tool for use with an machine diagnostic test. The machine diagnostic tool includes a precondition determiner, a setting verifier, and a preparatory formatter. The precondition determiner is configured to determine if a precondition is required for a subsequent machine diagnostic test. The setting verifier is configured to verify a current state of the precondition for the subsequent machine diagnostic test. The preparatory formatter is configured to provide instructions to make the precondition ready if required for the subsequent machine diagnostic test if the precondition is not ready for the subsequent machine diagnostic test.

There has thus been outlined, rather broadly, certain embodiments of the invention in order that the detailed description thereof herein may be better understood, and in order that the present contribution to the art may be better appreciated. There are, of course, additional embodiments of the invention that will be described below and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of embodiments in addition to those described and of being practiced and carried out in various ways. Also, it is to be understood that the phraseology and terminology employed herein, as well as the abstract, are for the purpose of description and should not be regarded as limiting.

As such, those skilled in the art will appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### BRIEF DESCRIPTION OF THE DRAWINGS

FIG. 1 illustrates an exemplary vehicle or machine diagnostic test setup of a type suitable for carrying out the functions of an embodiment of the invention.

FIG. 2 is a schematic diagram illustrating an operational state tracker according to a preferred embodiment of the invention.

FIG. 3 is a flowchart illustrating steps that may be followed to track the state of a vehicle or machine in accordance with one embodiment of the method or process.

FIG. 4 illustrates a representative state or status tracker according to another embodiment of the invention.

FIG. 5 is a flowchart illustrating method steps that may be followed in accordance with the embodiment of FIG. 4 for collecting and tracking the medical conditions in a patient.

### DETAILED DESCRIPTION

An embodiment of the present invention provides an operational state tracker that facilitates performance of vehicle or machine diagnostic test sequences by tracking the state of the subject vehicle or machine during and between individual diagnostic test procedures. The operational state tracker can help to eliminate duplication of efforts during a diagnostic test sequence by keeping track of the current vehicle or machine test configuration and providing test preparation steps to reconfigure the vehicle or machine between individual diagnostic procedures without redundant steps. The operational state tracker can track the current state of the vehicle or machine by maintaining a current list of preconditions, or vehicle or machine test configuration information.

The operational state tracker can include a precondition determiner that can determine the preconditions required for a subsequent diagnostic procedure, a state reader that can read a current state, for example, from a memory register, and a setting verifier than can verify the current setting of the operational state with regard to an individual precondition required for the diagnostic procedure. The operational state tracker can also include a test prep step formatter that can format a test preparation step related to a required precondition for display on a display device, if the precondition is required for the test procedure and the current setting of the operational state corresponding to the precondition is not valid. The operational state tracker can further include a feedback receiver that can receive feedback from a user, the vehicle, appliance, or test equipment indicating that the required precondition has been satisfied, and a state updater that can update the operational state, for example, in a memory register, to reflect the status of the precondition.

Similarly, the operational state tracker can determine a vehicle or machine condition that is not required for the subsequent diagnostic procedure, verify the current setting of the operational state with regard to the condition, and format a test preparation step that instructs the vehicle or machine technician to reverse the condition, if the condition is not required for the subsequent test procedure and the current setting is valid.

The terms “valid” and “invalid” as used in this disclosure regarding the operational state settings corresponding to preconditions describe the operational state setting. As used in this disclosure, the term “valid” means that the current setting indicates that the precondition is set, indicating that the precondition is currently met or the corresponding vehicle or machine test configuration is currently set up. Correspondingly, the terms “invalid” or “not valid” as used in this disclosure mean that the current operational state setting is not set, indicating that the corresponding precondition is not currently met or that the corresponding vehicle or machine test configuration is not currently set up.

An embodiment of the operational state tracker can complement or can be an integral part of a diagnostic test procedure generator. An example of a diagnostic test procedure generator that is compatible with the interactive diagnostic schematic generator is disclosed in copending U.S. Patent Application, entitled “Diagnostic Decision Sequencing Method and Apparatus for Optimizing a Diagnostic Test Plan,” filed concurrently herewith by Fountain, et al., the disclosure of which is hereby incorporated by reference in its entirety.

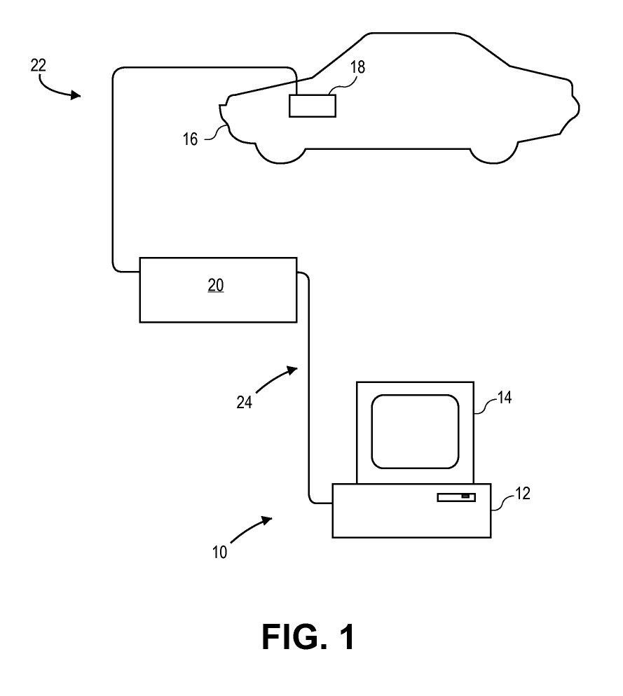

The invention will now be described with reference to the drawing figures, in which like reference numerals refer to like parts throughout. FIG. 1 illustrates a vehicle or machine test configuration that is compatible with the present inventive method and apparatus. An operational state tracker 10 can include a personal computer 12 with a display device 14. The operational state tracker 10 can be coupled to a machine 16, including, for example, an onboard computer 18. The machine 16 may include any suitable mechanical or electronic device such as a vehicle, appliance, tools, or the like. Suitable vehicles include, for example, cars, trucks, trains, boats, planes, all-terrain vehicles, and the like. Suitable appliances include, for example, washing machines and driers, dish washers, vacuums, microwave ovens, refrigerators, ice cream makers, and the like. Suitable tools include, for example, drills, saws, routers, sanders, measuring tools. fluid electronic timers, pneumatic tools, and the like. The operational state tracker 10 can be coupled to the onboard computer 18 by way of an interface box 20, as shown in FIG. 1. The vehicle or machine test configuration can further include electrical links 22, 24, such as wires, cables, data buses, a communication network or a wireless network. The operational state tracker 10 can display diagnostic test procedure instructions to a vehicle or machine technician to aid in performing vehicle or machine diagnostics. The operational state tracker 10 can also receive feedback from the machine 16.

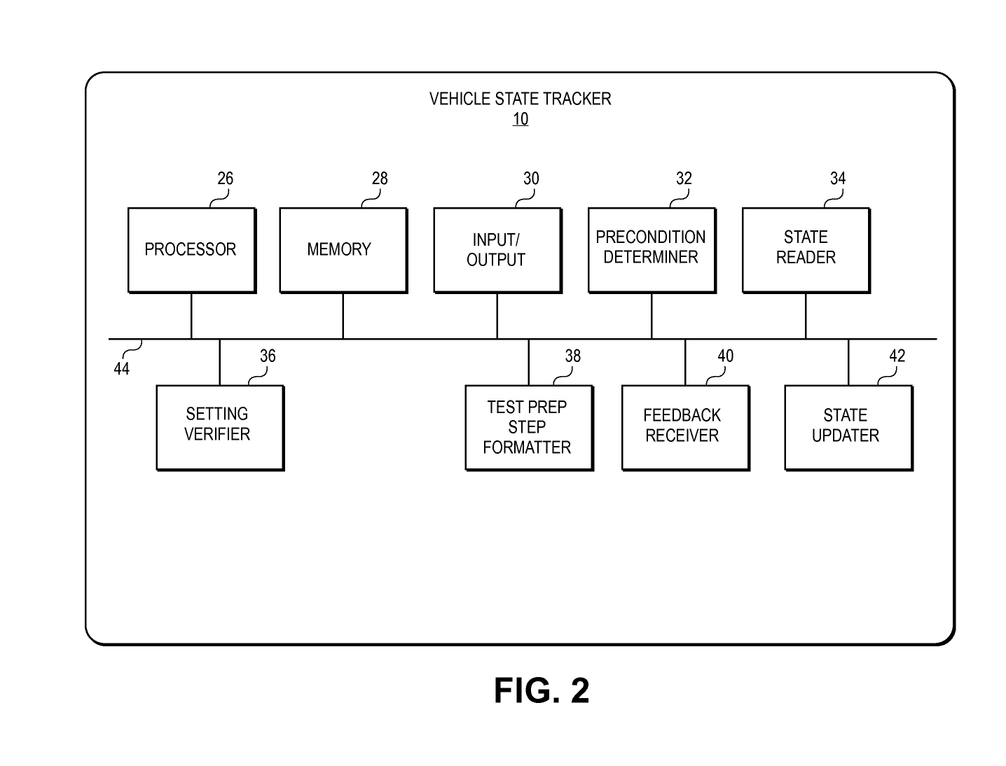

As illustrated in FIG. 2, an operational state tracker 10 can include a processor 26, a memory 28, an input/output device 30, a precondition determiner 32, a state reader 34, a setting verifier 36, a test preparation step formatter 38, a feedback receiver 40, and a state updater 42, all of which can be interconnected by a data link 44. The processor 12, the memory 14, the input/output device 16 and the display device 34 can be part of a general computer, such as a personal computer (PC), a UNIX workstation, a server, a mainframe computer, a personal digital assistant (PDA), a wearable mountable device, a tablet, or some combination of these. Alternatively, the processor 12, the memory 14 and the input/output device 16 can be part of a specialized computing device, such as a vehicle or machine diagnostics scan tool. The remaining components can include programming code, such as source code, object code or executable code, stored on a computer-readable medium that can be loaded into the memory 14 and processed by the processor 12 in order to perform the desired functions of the operational state tracker 10.

In various embodiments, the operational state tracker 10 can be coupled to a communication network, which can include any viable combination of devices and systems capable of linking computer-based systems, such as the Internet; an intranet or extranet; near field communication (NFC), a local area network (LAN); a wide area network (WAN); a direct cable connection; a private network; a public network; an Ethernet-based system; a token ring; a value-added network; a telephony-based system, including, for example, T1 or E1 devices; an Asynchronous Transfer Mode (ATM) network; a wired system; a wireless system; an optical system; a combination of any number of distributed processing networks or systems or the like.

An embodiment of the operational state tracker 10 can be coupled to the communication network by way of the local data link, which in various embodiments can incorporate any combination of devices—as well as any associated software or firmware—configured to couple processor-based systems, such as modems, network interface cards, serial buses, parallel buses, LAN or WAN interfaces, wireless or optical interfaces and the like, along with any associated transmission protocols, as may be desired or required by the design.

Additionally, an embodiment of the operational state tracker 10 can communicate information to the user and request user input by way of an interactive, menu-driven, visual display-based user interface, or graphical user interface (GUI). The user interface can be executed, for example, on a personal computer (PC) with a mouse and keyboard, with which the user may interactively input information using direct manipulation of the GUI. Direct manipulation can include the use of a pointing device, such as a mouse or a stylus, to select from a variety of selectable fields, including selectable menus, drop-down menus, tabs, buttons, bullets, checkboxes, text boxes, and the like. Nevertheless, various embodiments of the invention may incorporate any number of additional functional user interface schemes in place of this interface scheme, with or without the use of a mouse or buttons or keys, including for example, a trackball, a touch screen or a voice-activated system.

The precondition determiner 32 can determine a set of preconditions, or vehicle or machine test configuration requirements, necessary for an individual diagnostic test procedure. Preconditions and corresponding test preparation steps can be created, or authored, for example, by an expert diagnostics technician. Preconditions can also be formatted to be reusable in various diagnostic test procedures, which can save time during the authoring phase of diagnostic test procedures. In operation, the precondition determiner 32 typically can determine the preconditions required for a subsequent diagnostic test procedure before the completion of a current diagnostic test procedure in order to prevent or minimize redundant efforts at the completion of the current diagnostic procedure and at the initiation of the subsequent diagnostic procedure.

The state reader 34 can read a current state of the vehicle or machine, for example, from a memory register. In some embodiments, the operational state can be stored in a processor register, while in other embodiments the operational state can be stored in a main memory register or in a memory register of a storage device associated with the personal computer 12. The setting verifier 36 can verify a current setting of the operational state with regard to a specific precondition, or a group of current settings corresponding to a number of preconditions.

Regarding a precondition that is required for the subsequent test procedure, if the corresponding operational state setting is currently not valid, the test preparation step formatter 38 can format a test preparation step for display on the display device 14 to instruct the vehicle or machine technician to set up the required precondition or vehicle or machine test configuration. Of course, if the precondition is required for the subsequent test procedure and the corresponding operational state setting is currently valid, the test preparation step formatter 38 may elect not to format a test preparation step for display.

Thus, the test preparation step can be displayed to the vehicle or machine technician to instruct the technician to satisfy a required precondition for the diagnostic procedure. Correspondingly, if the precondition was required for the current diagnostic procedure and as also required for the subsequent diagnostic procedure, the vehicle or machine diagnostic system does not instruct the vehicle or machine technician to perform redundant vehicle or machine test configuration setup labor. As a result, the operational state tracker 10 can help eliminate repetitive steps, facilitating a faster and more accurate diagnosis of a vehicle or machine operational problem.

In addition, the operational state tracker 10 can include a feedback receiver 40 that can receive feedback indicating when the precondition has been satisfied. For example, the feedback receiver 40 can receive a data signal from the onboard computer 18 indicating that the precondition has been satisfied. Similarly, the feedback receiver 40 can receive a feedback signal from test equipment, such as a digital multimeter, coupled to the machine 16. Otherwise, the feedback receiver 40 can receive user input from the vehicle or machine technician by way of the input/output device 30 indicating that the precondition has been satisfied, or that the vehicle or machine technician has complied with the test preparation step instructions.

Once the precondition has been satisfied, the state updater 42 can update the operational state, for example, in a memory register, to reflect a valid setting corresponding to the precondition. Thus, the operational state can be continuously updated to maintain a current and accurate operational state that is available to the diagnostic system at any time in order to determine test preparation steps required to reconfigure the machine 16 between diagnostic procedures in a diagnostic test sequence.

In the case that the vehicle or machine condition is currently valid but is not required for a subsequent test procedure, the test preparation step formatter 38 can format a test preparation step for display instructing the vehicle or machine technician to reverse, or undo, the vehicle or machine condition. Correspondingly, the feedback receiver 40 can receive feedback as described above indicating that the condition has been reversed, and the state updater 42 can update the operational state, for example, in a memory register, to reflect an invalid setting corresponding to the condition, or precondition.

The operational state tracker 10 can maintain operational state settings for any number of vehicle or machine preconditions associated with the diagnostic test procedures. For example, preconditions can include the following: an ignition switch position; an engine run condition; a throttle position; an engine or motor speed; a vehicle or machine speed; a test equipment connection; a vehicle or machine electrical connection condition; an ambient air temperature; an engine or motor inlet temperature; an engine or motor lubricant pressure; an engine or motor lubricant temperature; an engine or motor lubricant level; an engine or motor coolant temperature; an engine or motor coolant specific gravity; an engine or motor exhaust gas temperature; an engine or motor exhaust gas content; a transmission setting; a brake pedal position; a parking brake position; a brake fluid pressure; a fuel level; a fuel supply pressure; a battery voltage; a battery charging system voltage; a battery charging system current; an ignition voltage; an ignition current; an engine cylinder compression; a pyrotechnic actuator condition; a saw blade connection; a riving knife connection; a kick-back detection system condition; a sensing detecting system condition; blade or arbor sensor signal; a vehicle or machine configuration; or a vehicle or machine modification.

As an operational example, in preparation for performing a diagnostic procedure on a vehicle to determine if a throttle position sensor (TPS) terminal wire is shorted to the battery positive voltage source, the precondition determiner 32 may determine that the following four preconditions must be met: ignition switch “on”; TPS connector disconnected; voltmeter (VOM) red lead connected to TPS connector socket; and voltmeter (VOM) black lead connected to electrical ground. Similarly, a machine may include a variable position switch or trigger to control the speed of the machine. In preparation for performing a diagnostic procedure on the machine to determine if the variable position switch (VPS) terminal wire is shorted to the battery positive voltage source, the precondition determiner 32 may determine that the following four preconditions must be met: power switch “on”; VPS connector disconnected; voltmeter (VOM) red lead connected to VPS connector socket; and voltmeter (VOM) black lead connected to electrical ground.

The state reader 34 may then read the current state of the vehicle from a memory register, and the setting verifier 36 may determine that the ignition switch is currently “on” (valid), but that the remaining three preconditions are currently not valid. As a result, the test preparation step formatter 38 can format a graphical user interface window for display on a display device with the following test preparation steps: Disconnect TPS; Connect VOM red lead to TPS connector socket; and Connect VOM black lead to battery negative post. Similarly, when testing a machine, the state reader 34 may then read the current state of the machine from a memory register, and the setting verifier 36 may determine that the VPS is currently “on” (valid), but that the remaining three preconditions are currently not valid. As a result, the test preparation step formatter 38 can format a graphical user interface window for display on a display device with the following test preparation steps: Disconnect VPS; Connect VOM red lead to VPS connector socket; and Connect VOM black lead to battery negative post.

After completing the required test preparation steps, the vehicle or machine technician can provide a user input by way of the input/output device, such as a keyboard, a mouse, or a stylus, to indicate that the test preparation steps have been completed. The user input can be received by the feedback receiver 40, and in response, the state updater 42 can write the new operational state including the three newly valid preconditions to the memory register.

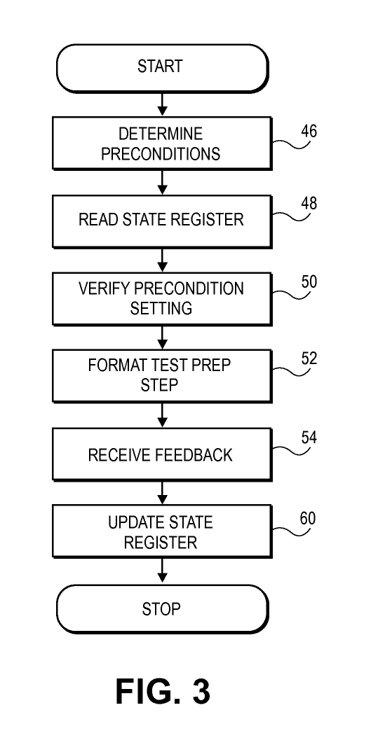

FIG. 3 is a flowchart illustrating a sequence of steps that can be performed in order to track the state of a vehicle or machine during a diagnostic test sequence. The process can begin by proceeding to step 46, “Determine Preconditions,” in which a precondition or a group of preconditions required for an individual diagnostic test procedure can be determined. As described above, preconditions typically are determined for a subsequent test procedure that is to follow a current procedure that has not yet been completed, in order to avoid repetitive steps during the transition from the current diagnostic procedure to the subsequent diagnostic procedure.

Then, in step 48, “Read State Register,” an operational state can be read, for example, from a memory register. As described above, the operational state can be stored in a processor register, a main memory or a peripheral storage device. After the preconditions have been determined and the current operational state has been read, in step 50, “Verify Precondition Setting,” the current setting of the operational state corresponding to a specific precondition can be verified. For example, an individual bit in a memory register can be verified.

If the precondition is required for the subsequent diagnostic test procedure and the current operational state setting corresponding to the precondition is not valid, in step 52, “Format Test Preparation Step,” a test preparation step can be formatted for display on a display device instructing the vehicle or machine technician to satisfy the precondition, or set up the corresponding vehicle or machine condition. Otherwise, if the precondition is not required for the subsequent diagnostic procedure and the current operational state setting corresponding to the precondition is valid, a test preparation step can be formatted instructing the vehicle or machine technician to reverse the vehicle or machine condition corresponding to the precondition.

Next, in step 54, “Receive Feedback,” feedback can be received indicating either that the precondition has been satisfied or that the corresponding vehicle or machine condition has been reversed. As described above, the feedback can be received as a data signal from the onboard computer, a test equipment signal from test equipment coupled to the vehicle, appliance, or user input.

At this point, in step 60, “Update State Register,” the operational state setting corresponding to the precondition can be updated to indicate either that the precondition has been satisfied (valid) or that the corresponding vehicle or machine condition has been reversed (invalid). In this way, the current and accurate state of the vehicle or machine configuration and conditions can be maintained, for example, in a memory register. Tracking the current operational state by this process can facilitate efficient vehicle or machine diagnostic testing by eliminating or minimizing duplicative efforts between diagnostic test steps in a diagnostic test sequence.

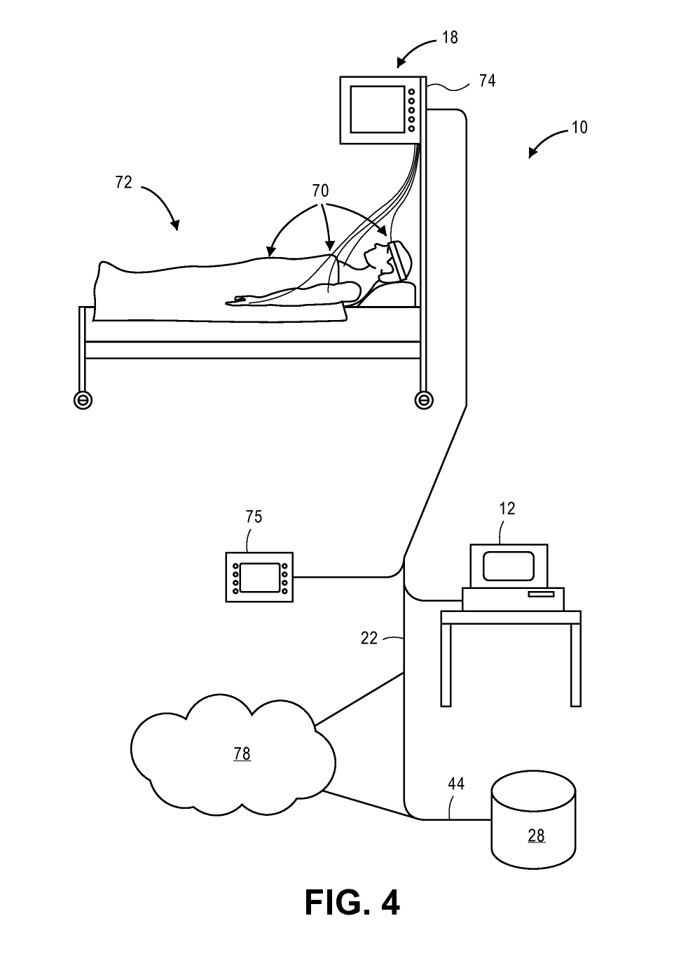

FIG. 4 illustrates a representative operational state tracker 10 according to another embodiment of the invention. In this embodiment, the operational state tracker 10 is utilized to track the medical status of a patient and so, may be described as a patient status tracker. The embodiment of FIG. 4 is similar to the embodiment of FIG. 1 and thus, in the interest of brevity, those elements described hereinabove will not be described again. As shown in FIG. 4, the patient status tracker 10 is configured to collect and/or analyze medical data. In this regard, the medical data analyzed may be obtained from one or more sensors 70 configured to sense vital signs or other such medically related data from a patient 72. These sensed vital signs may be collected, stored, analyzed, and/or displayed on a patient monitor 74. In addition, the patient status tracker 10 can include the PC 12 and/or the hand-held diagnostic scan tool 75 configured to be coupled to the patient 72 via the patient monitor 74. The patient monitor 74 can include an onboard computer 18 that can be accessed by way of electrical links 22, such as such as conductors, wires, cables, data buses, a communication network or a wireless network. The patient status tracker 10 is configured to aid a technician in identifying potential medical conditions in the patient 72.

The patient status tracker 10 can further include a database or the memory 28 coupled to the PC 12 or scan tool 75, for example, by way of local links 44 and a communication network 78. In an alternative embodiment, the memory 28 can be stored directly in a memory associated with the personal computer 12 or the scan tool 75. In this manner, suitable patient data may be store, accessed, analyzed, and/or displayed. Examples of suitable patient data include blood counts, results of tests and other such lab results, family history, and the like.

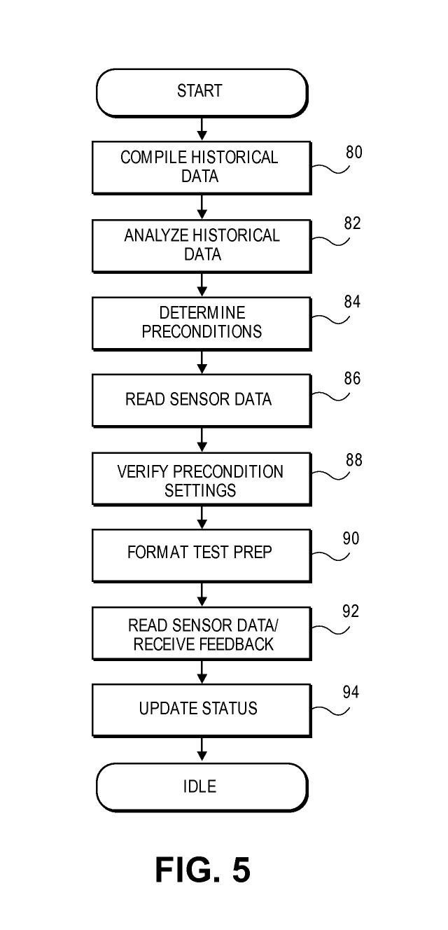

FIG. 5 is a flowchart illustrating method steps that may be followed in accordance with the embodiment of FIG. 4 for collecting and analyzing diagnostic data including historical data to diagnose medical conditions in a patient. The embodiment of FIG. 5 is similar to the embodiment of FIG. 3 and thus, in the interest of brevity, those steps described hereinabove will not be described again. As shown in FIG. 5, historical and/or sensed medical data of the patient 72 may be utilized to determine a set of preconditions, vital signs or other such medical data may be acquired and the status of the patient 72 may be updated based on the acquired data. In addition, the following method may be utilized to evaluate the effectiveness of a procedure or treatment program for the patient 72. In general, the patient 72 may be ‘challenged’ with a procedure that induces their precondition and, by comparing the current response to the challenge to the previous responses to the challenge, the procedure or treatment program may be evaluated.

The process can begin by proceeding to step 80, “Compile Historical Data,” wherein historical medical diagnostic data samples corresponding to various normal vital signs and disease conditions are gathered and organized. In addition, the historical data may include personal historical medical data from the patient such as, for example, previous vital signs, previous lab results, family history, and the like.

The historical diagnostic data can include various sensed vital signs and/or other medical data from a statistically significant population of individuals that are healthy and a likewise statistically significant population of diseased individuals. Furthermore, the historical data can be collected as a “snapshot”—a single set of measurements at a moment in time—or as a “data strip”—a sequence or series of periodic measurements taken over a period of time. This data can have all personal information removed to prevent identification of participants in the medical sampling.

The data can be accumulated in a database, such as a relational database that associates each instance of measured parameters with a definition or description of pre-existing and/or exacerbating conditions under which the data were gathered. In some embodiments, the data can be sent to a central repository, for example, over a communication network. In addition, known preconditions or challenges may be associated with various diseases. In a particular example, exercise may be used to challenge a patient with a history of exercise induced arrhythmia. As such, exercise or other such stress test may be considered a precondition for performing a test for exercise induced arrhythmia. Similarly, exercise may be a precondition for a number of other tests such as, for example, oxygen uptake (VO₂ max), cardio/pulmonary output, and the like. In another example, many tests are preferably performed while the patient is in an upright posture. By conducting all such medical tests at the same time or in succession, the patient and the technician time and money may be saved.

Then, in step 82, “Analyze Historical Data,” the historical diagnostic data samples can be analyzed to determine typical ranges for the vital signs or other such medical data corresponding to various normal and diseased conditions. In this step, a diagnostic case history can be defined, for example, as an ordered list of diagnostic procedures performed and associated preconditions the patient 72 is subjected to in order to perform the diagnostic procedure. In general, the analysis of the historical data may result in one or more particular precondition for performing diagnostic procedures specifically for the patient 72. In this manner, the specific medical procedures for diagnosis or identification of diseases, may be quickly and efficiently performed on the patient 72.

At step 84, a precondition may be determined. For example, based on the list generated by the analysis of the data, one or more preconditions may be determined. In a particular example, a first precondition of the preconditions from the list of preconditions may be initially selected. In other examples, all or some of the preconditions from the list of preconditions may be initially selected. An example of a specific medical precondition may include: fasting, standing, sitting, laying down, exercising, sleeping, and the like. In general, these and other preconditions may be associated with test for high blood pressure, diabetes, heart failure, cancer, Alzheimer's disease, sleep apnea, coma, liver failure, exercise induced arrhythmia, poisoning, trauma, and the like.

At step 86, a current medical status may be determined. For example, the sensors 70 may be utilized to determine the current medical status of the patient 72. For example, a blood pressure of the patient 72 may be read. At step 88, the precondition may be verified. For example, if exercise is the precondition selected at step 84 and the patient 72 is exercising, then the precondition may be verified. If verified, some or all the tests associated with the precondition of exercise may be performed at step 90. Alternatively, if the patient 72 is not performing the precondition of exercise, this precondition may be flagged or removed as a precondition.

If the precondition is required for the subsequent diagnostic test procedure and the current status of the patient corresponding to the precondition is not valid, at step 90, “Format Test Preparation,” a test preparation can be formatted for display on a display device instructing the technician or medical staff to satisfy the precondition. For example, if the precondition is exercise to induced arrhythmia, the technician may set up a stress test for the patient 72. Otherwise, if the precondition is not required for the subsequent diagnostic procedure and the current medical status of the patient 72 corresponding to the precondition is valid, a test preparation can be formatted instructing the technician to stop the precondition.

Next, at step 92, “Read Sensor Data/Receive Feedback,” feedback can be received indicating either that the precondition has been satisfied. As described above, the feedback can be received as a data signal from the onboard computer 18, a test equipment signal from the sensors 70 coupled to the patient 72, an indicator from the technician, or the like.

At this point, at step 94, “Update Status,” the patient medical status corresponding to the precondition can be updated to indicate either that the precondition has been satisfied (valid) or that the precondition has not been satisfied (invalid). In this way, the current and accurate medical status of the patient 72 and conditions can be maintained, for example, in a memory register at a local computer or a remote computing device, for example. Tracking the current medical status of the patient 72 by this process can facilitate efficient medical diagnostic testing by eliminating or minimizing duplicative efforts between diagnostic test steps in a diagnostic test sequence. More particularly, identifying the precondition of the patient 72 allows all tests associated with the precondition to be performed at once or in succession in order to increase efficiency.

Of note, while the above embodiment is described with particular focus on medical preconditions and associated medical test or procedures to be performed upon the patient 72, other embodiments need not be so directed, but rather, may be directed towards testing of appliances, devices and/or machinery. For example, while testing a refrigeration unit in an air conditioning (A/C) unit, various suitable preconditions may be tested for. Examples of suitable preconditions may include, running, stopped, de-powered, fan only, full load, and the like.

FIGS. 2, 3 and 5 are block diagrams and flowcharts of methods, apparatuses and computer program products according to various embodiments of the present invention. It will be understood that each block or step of the block diagram, flowchart and control flow illustrations, and combinations of blocks in the block diagram, flowchart and control flow illustrations, can be implemented by computer program instructions or other means. Although computer program instructions are discussed, an apparatus according to the present invention can include other means, such as hardware or some combination of hardware and software, including one or more processors or controllers, for performing the disclosed functions.

In this regard, FIG. 2 depicts the apparatus of one embodiment including several of the key components of a general purpose computer by which an embodiment of the present invention may be implemented. Those of ordinary skill in the art will appreciate that a computer can include many more components than those shown in FIG. 2. However, it is not necessary that all of these generally conventional components be shown in order to disclose an illustrative embodiment for practicing the invention. The general purpose computer can include a processing unit 26 and a system memory 28, which may include random access memory (RAM) and read-only memory (ROM). The computer also may include nonvolatile storage memory, such as a hard disk drive, where additional data can be stored.

An embodiment of the present invention can also include one or more input or output devices 30, such as a mouse, keyboard, monitor, and the like. A display can be provided for viewing text and graphical data, as well as a user interface to allow a user to request specific operations. Furthermore, an embodiment of the present invention may be connected to one or more remote computers via a network interface. The connection may be over a local area network (LAN), wide area network (WAN), the Internet, or the like, and can include all of the necessary circuitry for such a connection.

Typically, computer program instructions may be loaded onto the computer or other general purpose programmable machine to produce a specialized machine, such that the instructions that execute on the computer or other programmable machine create means for implementing the functions specified in the block diagrams, schematic diagrams or flowcharts. Such computer program instructions may also be stored in a computer-readable medium that when loaded into a computer or other programmable machine can direct the machine to function in a particular manner, such that the instructions stored in the computer-readable medium produce an article of manufacture including instruction means that implement the function specified in the block diagrams, schematic diagrams or flowcharts.

In addition, the computer program instructions may be loaded into a computer or other programmable machine to cause a series of operational steps to be performed by the computer or other programmable machine to produce a computer-implemented process, such that the instructions that execute on the computer or other programmable machine provide steps for implementing the functions specified in the block diagram, schematic diagram, flowchart block or step.

Accordingly, blocks or steps of the block diagram, flowchart or control flow illustrations support combinations of means for performing the specified functions, combinations of steps for performing the specified functions and program instruction means for performing the specified functions. It will also be understood that each block or step of the block diagrams, schematic diagrams or flowcharts, as well as combinations of blocks or steps, can be implemented by special purpose hardware-based computer systems, or combinations of special purpose hardware and computer instructions, that perform the specified functions or steps.

As an example, provided for purposes of illustration only, a data input software tool of a search engine application can be a representative means for receiving a query including one or more search terms. Similar software tools of applications, or implementations of embodiments of the present invention, can be means for performing the specified functions. For example, an embodiment of the present invention may include computer software for interfacing a processing element with a user-controlled input device, such as a mouse, keyboard, touch screen display, scanner, or the like. Similarly, an output of an embodiment of the present invention may include, for example, a combination of display software, video card hardware, and display hardware. A processing element may include, for example, a controller or microprocessor, such as a central processing unit (CPU), arithmetic logic unit (ALU), or control unit.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

What is claimed is:

1. A medical diagnostic tool for use with a medical diagnostic test, comprising:
a precondition determiner configured to determine if a precondition is required for a subsequent medical diagnostic test;
a setting verifier configured to verify a current state of the precondition for the subsequent medical diagnostic test; and
a preparatory formatter configured to provide instructions to make the precondition ready if required for the subsequent medical diagnostic test if the precondition is not ready for the subsequent medical diagnostic test.

2. The medical diagnostic tool of claim 1, wherein the instructions are provided on a display of the medical diagnostic tool.

3. The medical diagnostic tool of claim 1 further comprising:
a state reader configured to read the state of the precondition.

4. The medical diagnostic tool of claim 1 further comprising:
a state updater configured to update a register on whether the precondition is now ready or not ready.

5. The medical diagnostic tool of claim 1 further comprising:
a feedback receiver configured to receive a feedback that the precondition has been satisfied for the subsequent medical diagnostic test.

6. The medical diagnostic tool of claim 5, wherein the feedback is from an onboard computer data, a test equipment data or a technician's input.

7. The medical diagnostic tool of claim 1, wherein the preparatory step formatter does not provide instructions if the precondition is already ready for the subsequent medical diagnostic test.

8. The medical diagnostic tool of claim 1, wherein if the precondition determiner determines that the precondition is ready but not required for the subsequent medical diagnostic test, then the preparatory step formatter provides a reverse step to a user to make the precondition into a not ready state.

9. A computer implemented method of tracking a state of a precondition, comprising the steps of:
determining the precondition required for a subsequent medical diagnostic test, via a processor of the computer;
determining if the required precondition is ready for the subsequent medical diagnostic test, via the processor; and
providing instructions to a user to make the required precondition ready if the precondition is not ready, via the processor.

10. The computer implemented method of claim 9 further comprising the step of:
providing instructions to the user to make the precondition not ready if the precondition is not required by the subsequent medical diagnostic test.

11. The computer implemented method of claim 9 further comprising the step of:
verifying that all of the preconditions required for the subsequent medical diagnostic test are ready.

12. The computer implemented method of claim 11 further comprising the step of:
updating a state register to reflect that all the preconditions required for the subsequent medical diagnostic test are ready.

13. The computer implemented method of claim 11, wherein the verifying step uses information from an onboard computer data, a test equipment data or a technician input.

14. A machine diagnostic tool for use with a machine diagnostic test, comprising:
a precondition determiner configured to determine if a precondition is required for a subsequent machine diagnostic test;
a setting verifier configured to verify a current state of the precondition for the subsequent machine diagnostic test; and
a preparatory formatter configured to provide instructions to make the precondition ready if required for the subsequent machine diagnostic test if the precondition is not ready for the subsequent machine diagnostic test.

15. The machine diagnostic tool of claim 14, wherein the instructions are provided on a display of the machine diagnostic tool.

16. The machine diagnostic tool of claim 14 further comprising:
a state reader configured to read the state of the precondition.

17. The machine diagnostic tool of claim 14 further comprising:
a state updater configured to update a register on whether the precondition is now ready or not ready.

18. The machine diagnostic tool of claim 14 further comprising:
a feedback receiver configured to receive a feedback that the precondition has been satisfied for the subsequent machine diagnostic test.

19. The machine diagnostic tool of claim 18, wherein the feedback is from an onboard computer data, a test equipment data or a technician's input.

20. The machine diagnostic tool of claim 14, wherein the preparatory step formatter does not provide instructions if the precondition is already ready for the subsequent machine diagnostic test.
